(19) United States
(12) Patent Application Publication
(10) Pub. No.: US 2026/0355721 A1
(43) Pub. Date: Nov. 12, 2026

(54) EPOXIDATION CATALYST

(71) Applicant: BASF SE, Ludwigshafen am Rhein (DE)

(72) Inventors: Andrey Karpov, Ludwigshafen am Rhein (DE); Sung Yeun CHOI, Ludwigshafen (DE); Phil Jack HOLZMEISTER, Mannheim (DE); Christian WALSDORFF, Ludwigshafen am Rhein (DE); Christian BARTOSCH, Ludwigshafen am Rhein (DE)

(21) Appl. No.: 19/120,249

(22) PCT Filed: Oct. 12, 2023
(86) PCT No.: PCT/EP2023/078309

§ 371 Date: Apr. 11, 2025

(30) Foreign Application Priority Data
Oct. 12, 2022 (EP) 22201043.1

(51) Int. Cl. B01J 23/68; B01J 21/12; B01J 35/30; B01J 35/61; B01J 35/63; B01J 37/02; B01J 37/08; C07D 301/10

(52) U.S. Cl. CPC B01J 23/688; B01J 21/12; B01J 35/394; B01J 35/612; B01J 35/633; B01J 35/635; B01J 37/0213; B01J 37/08; C07D 301/10

(57) ABSTRACT

An epoxidation catalyst comprises silver, cesium, rhenium and tungsten deposited on a low silicon alpha-alumina support which comprises at least 50 wt.-% of alpha-alumina, and an amount of silicon c_{S-Si} of at most 8.9 mmol per kg of support, and the catalyst further comprises potassium and optionally sodium. The catalyst comprises 25 to 50 wt.-% of silver, an amount of cesium c_{Cs} of at least 6.0 mmol per kg of catalyst, an amount of rhenium C_{Re} of at least 6.0 mmol per kg of catalyst, an amount of tungsten C_{W} of at least 3.0 mmol per kg of catalyst, an amount of potassium C_{K} of at least 3.0 mmol per kg of catalyst, and an amount of sodium C_{Na} of at most 4.0 mmol per kg of catalyst. The catalyst has a value of P_{A} (Formula (I)) in the range of 0.62 to 0.76. The catalyst allows for a more efficient conversion of ethylene oxide by gas-phase oxidation of ethylene, displaying high initial selectivity and activity as well as maintenance of selectivity over time. A process for preparing the epoxidation catalyst comprises i) impregnating a low silicon alpha-alumina support with a silver impregnation solution and ii) subjecting the impregnated refractory support to a calcination process, wherein steps i) and ii) are optionally repeated, and at least one silver impregnation solution comprises rhenium, tungsten, cesium, potassium, and optionally sodium.

The present invention relates to a catalyst effective in the oxidative conversion of ethylene to ethylene oxide, a process for preparing the catalyst, and a process for preparing ethylene oxide by gas-phase oxidation of ethylene by means of oxygen in the presence of the catalyst.

Ethylene oxide is produced in large volumes and is primarily used as an intermediate in the production of several industrial chemicals. For the industrial oxidation of ethylene to ethylene oxide, heterogeneous catalysts comprising metallic silver are used. Catalyst performance may be characterized, e.g., by selectivity, activity, longevity of catalyst selectivity and/or longevity of catalyst activity. Selectivity is the molar fraction of the converted olefin yielding the desired olefin oxide. Even small improvements in selectivity and the maintenance of selectivity over longer time yield huge dividends in terms of process efficiency.

Suitable epoxidation catalysts are generally obtained by depositing metallic silver on a support. Highly selective silver-based epoxidation catalysts have been developed, which extend the selectivity to a value that is closer to the stoichiometric limit. Such highly selective catalysts comprise, in addition to silver as the active component, promoting species for improving the catalytic properties of the catalyst, as described in, e.g., WO 2007/122090 A2 and WO 2010/123856 A1. Examples of promoting species include alkali metal compounds and/or alkaline earth metal compounds, as well as transition metals such as rhenium, tungsten or molybdenum.

Supports for epoxidation catalysts are characterized by both their chemical composition and their physical properties, such as surface area and porosity. Both the chemical composition and the physical properties of a support impact the performance of catalysts based thereon and often require different formulations of promoting species deposited thereon to achieve the best catalyst performance.

Alumina (Al₂O₃) is ubiquitous in supports and/or catalysts for many heterogeneous catalytic processes. Some of these catalytic processes occur under conditions of high temperature, high pressure and/or high water-vapor pressure. It is well known that alumina has a number of crystalline phases such as alpha-alumina (often denoted as α-alumina or α-Al₂O₃), gamma-alumina (often denoted as γ-alumina or γ-Al₂O₃) as well as a number of alumina polymorphs. alpha-Alumina is the most stable at high temperatures, but has the lowest surface area.

In the process of making a catalyst support, the alpha-alumina phase is typically mixed with temporary and permanent binders. The temporary binders are thermally decomposable organic compounds of moderate to high molecular weight which, on decomposition, produce the pore structure of the support. The permanent binders are inorganic clay-type materials having fusion temperatures below that of the alumina and are responsible for imparting mechanical strength to the finished support. Silica can also be added in quantity sufficient to obtain a finished support of the desired strength and composition. For instance, EP 1 955 766 A1 describes an epoxidation catalyst comprising a carrier with a silicon content of 0.5 to 7.0 wt.-% in terms of silica. Generally, the silicon thus incorporated into the support must be accounted for in the formulations of promoting species deposited thereon to achieve the best catalyst performance.

WO 2018/029189 A1 describes an ethylene oxide catalyst comprising silver, cesium and rhenium applied to an alumina support. The support comprises silicon, calcium and magnesium, with the amount of silicon exceeding the total amount of calcium and magnesium.

WO 2019/154832 A1 describes a catalyst effective in the oxidative conversion of ethylene to ethylene oxide, comprising an alumina support and silver applied to the support, wherein the catalyst comprises defined amounts of cesium, rhenium, tungsten and a specific silicon to earth metal molar ratio.

WO 2021/260138 A1 describes a shaped catalyst body for ethylene oxide production comprising silver and a rhenium promoter deposited on a porous alpha-alumina support, characterized in that the support has a calcination history of at least 1460 °C.

WO 2021/260182 A1 describes a process for producing a porous alpha-alumina catalyst support via a precursor material comprising at least 50 wt.-% of a transition alumina with specific loose bulk density and porosity. Through the use of transition alumina as a starting material, permanent binders such as silicates or silicon dioxide can be dispensed with, resulting in low silicon binders.

WO 2021/260185 A1 describes a tableted alpha-alumina catalyst support. The tableted support has high geometrical precision and may be obtained from high purity transition aluminas which limit the content of impurities such as sodium or silicon in the support.

WO 2022/161924 A1 describes an epoxidation catalyst comprising silver, cesium, rhenium, tungsten deposited on an alumina support, wherein the amount of cesium, the amount of rhenium and the combined amounts of rhenium and tungsten are specified.

To obtain a catalyst having high activity and high selectivity for the production of ethylene oxide, the combination of catalyst ingredients and support properties is important. The raw material of support production is usually natural minerals, which inevitably contain impurities, such as alkali metals, in particular sodium or potassium. The impurities are not lost in the manufacturing process and are finally found in the catalyst support and catalyst. This has unreliable effects on the performance of the catalyst. Therefore, the alkali metal content must be systematically considered in the process of catalyst preparation.

Moreover, potential interactions amongst promoting species, as well as amongst impurities and between promoting species and impurities must be considered in the process of catalyst preparation. The development of an effective catalyst for catalyst supports is thus far from trivial.

There remains a significant need for an epoxidation catalyst based on a support with low silicon content, which catalyst allows for a more efficient conversion of ethylene oxide by gas-phase oxidation of ethylene, particularly a catalyst displaying high initial selectivity and activity as well as maintenance of selectivity over time.

The invention relates to an epoxidation catalyst comprising silver, cesium, rhenium and tungsten deposited on a low silicon alpha-alumina support which comprises at least 50 wt.-% of alpha-alumina, and an amount of silicon c_{S-Si} of at most 8.9 mmol per kg of support, the catalyst further comprising potassium and optionally sodium, wherein the catalyst comprises
25 to 50 wt.-% of silver, relative to the weight of the catalyst,
an amount of cesium c_{Cs} of at least 6.0 mmol per kg of catalyst,
an amount of rhenium c_{Re} of at least 6.0 mmol per kg of catalyst,
an amount of tungsten c_{W} of at least 3.0 mmol per kg of catalyst,
an amount of potassium c_{K} of at least 3.0 mmol per kg of catalyst, and
an amount of sodium c_{Na} of at most 4.0 mmol per kg of catalyst; and
wherein the value of P_{A}

$${P}_{A} = \frac{{c}_{Re} + ( 0.5 \times {c}_{W} )}{( 1.5 \times {c}_{Cs} ) + ( 0.5 \times ( {c}_{K} + {c}_{N a} ) )}$$

is in the range of 0.62 to 0.76;
the elemental composition of the catalyst support and the catalyst being determined by elemental analysis via inductively coupled plasma optical emission spectroscopy.

It was found that alpha-alumina supports with low amounts of silicon require specific optimum amounts of promoting species, and moreover that the optimum amounts of the promoting species are to some extent interdependent. The synergistic effect observed by choosing the amounts of rhenium c_{Re} and tungsten c_{W}, as well as the amounts of cesium c_{Cs}, potassium c_{K} and sodium c_{Na} so as to fall within the specified range of P_{A} allows for a catalyst with an especially high selectivity.

Without wishing to be bound by theory, it is believed that rhenium and tungsten are to some extent interchangeable since both elements may exist in a form coordinated four-fold or six-fold by oxygen, i.e. as perrhenate and tungstate; and that cesium is to some extent interchangeable with potassium and sodium, which are likewise alkali metals. The formula contains factors determining the rate at which a portion of rhenium may be substituted by tungsten, or the rate at which a portion of cesium may be substituted by sodium and/or potassium.

The value of P_{A}

$${P}_{A} = \frac{{c}_{Re} + ( 0.5 \times {c}_{W} )}{( 1.5 \times {c}_{Cs} ) + ( 0.5 \times ( {c}_{K} + {c}_{N a} ) )}$$

of the catalyst is in the range of 0.62 to 0.76, preferably 0.62 to 0.72, more preferably 0.64 to 0.70. Inferior results may be achieved if the value of P_{A} is outside these ranges.

It was moreover found that the ratio of the total amounts of potassium c_{K} and sodium c_{Na} to the amount of cesium c_{Cs} impacts the stability of the catalyst selectivity over time. Preferably, the value of P₈

$${P}_{B} = \frac{{c}_{K} + {c}_{N a}}{{c}_{Cs}}$$

is in the range of 0.62 to 0.94, preferably 0.63 to 0.90, more preferably 0.64 to 0.90. It was found that a value of P₈ in this range allows for an especially high selectivity stability.

The alkali metals may be present in the catalyst as oxides, hydroxides or as counterions of oxyanions of other promoting elements. Without wishing to be bound by theory, it is believed that a combination of potassium and sodium with cesium within the ranges defined by P_{B} may control the mobility of alkali metals, helping to maintain a balance of promoting species on the silver and on the support surface during catalyst operation.

The catalyst comprises 25 to 50 wt.-% of silver, relative to the weight of the catalyst. Preferably, the catalyst comprises 26 to 40 wt.-% of silver, relative to the weight of the catalyst. Most preferably, the catalyst comprises 27 to 35% of silver, relative to the weight of the catalyst. A silver content in this range allows for a favorable balance between turnover induced by the catalyst and cost-efficiency of producing the catalyst.

The catalyst comprises an amount of cesium c_{Cs} of at least 6.0 mmol per kg of catalyst. It is particularly preferred that the catalyst comprises an amount of cesium c_{Cs} of 6.0 to 9.0 mmol per kg of catalyst, especially 6.0 to 8.5 mmol per kg of catalyst.

The catalyst comprises an amount of rhenium c_{Re} of at least 6.0 mmol per kg of catalyst. Preferably, the catalyst comprises an amount of rhenium c_{Re} of 6.0 to 9.0 mmol per kg of catalyst, especially 6.0 to 8.0 mmol per kg of catalyst.

The catalyst comprises an amount of tungsten c_{W} of at least 3.0 mmol per kg of catalyst. Preferably, the catalyst comprises an amount of tungsten c_{W} of 3.0 to 5.0 mmol per kg of catalyst, especially 3.0 to 4.0 mmol per kg of catalyst.

The catalyst comprises an amount of potassium c_{K} of at least 3.0 mmol per kg of catalyst. Preferably, the catalyst comprises an amount of potassium c_{K} of 3.0 to 6.0 mmol per kg of catalyst, more preferably 3.0 to 5.0, especially 3.0 to 4.5 mmol per kg of catalyst.

The catalyst comprises an amount of sodium c_{Na} of at most 4.0 mmol per kg of catalyst. Preferably, the catalyst comprises an amount of sodium c_{Na} of at most 3.0 mmol per kg of catalyst, especially 0.5 to 2.2 mmol per kg of catalyst.

The support is a low silicon alpha-alumina support, as described in detail below. Preferably, no silicon is applied during preparation of the catalyst. Without wishing to be bound by theory, e.g., it is believed that silicon induces acidic groups on the surface of the carrier, which promote the undesired conversion of ethylene oxide to acetaldehyde, thereby lowering the selectivity of the epoxidation process.

The catalyst preferably comprises an amount of silicon c_{Si} of at most 6.7 mmol per kg of catalyst, preferably of at most 5.4 mmol per kg of catalyst, more preferably of at most 4.0 mmol per kg of catalyst. Due to the nature of the raw materials generally used for obtaining alpha-alumina supports, the support typically may inherently comprise silicon, potassium and/or sodium. The catalyst may comprise an amount of silicon c_{Si} of 0.001 mmol per kg of catalyst or more, 0.01 mmol per kg of catalyst or more, 0.1 mmol per kg of catalyst or more, or 0.25 mmol per kg of catalyst or more.

The amount of silicon c_{Si} is understood to relate to the total amount of silicon in the catalyst, i.e., the amount of silicon added to the support via, e.g., impregnation, and the amount of silicon comprised in the support per se. Moreover, the amount of potassium c_{K} is understood to relate to the total amount of potassium in the catalyst, i.e., the amount of potassium added to the support via, e.g., impregnation, and the amount of potassium comprised in the support per se. Likewise, the amount of sodium c_{Na} is understood to relate to the total amount of sodium in the catalyst, i.e., the amount of sodium added to the support via, e.g., impregnation, and the amount of sodium comprised in the support per se.

The elemental composition of the catalyst support, as well as the elemental composition of the catalyst and the starting materials used for obtaining the catalyst support, may be determined by elemental analysis via inductively coupled plasma optical emission spectroscopy (ICP-OES), by Flame Atomic Absorption Spectroscopy (F-AAS) or by other established methods, in particular by inductively coupled plasma optical emission spectroscopy (ICP-OES). In order to obtain accurate results of total weight contents of impurities, samples of alumina supports should be fully dissolved and analysis performed on the solutions. A suitable method for fully dissolving alumina supports is described in Method 1 below.

In some embodiments, the catalyst may include a promoting amount of an alkali metal besides cesium, potassium and sodium (an “additional alkali metal”) or a mixture of two or more of such alkali metals, such as lithium or rubidium, or combinations thereof.

If lithium is employed as an additional alkali metal, then the amount of lithium will typically range from 14.0 to 100 mmol per kg of catalyst, more typically 40.0 to 100 mmol per kg of catalyst.

If rubidium is employed as an additional alkali metal, then the amount of rubidium will typically be at most of 10.0 mmol per kg of catalyst, more typically at most 7.0 mmol per kg of catalyst, relative to the total weight of the catalyst, most typically at most 4.0 mmol per kg of catalyst. The catalyst may comprise the additional alkali metal in an amount of 0.001 mmol per kg of catalyst or more, 0.01 mmol per kg of catalyst or more, 0.1 mmol per kg of catalyst or more, or 0.25 mmol per kg of catalyst or more.

Preferably, the additional alkali metal is lithium.

The catalyst may also include a Group IIA alkaline earth metal or a mixture of two or more Group IIA alkaline earth metals. Suitable alkaline earth metal promoters include, for example, beryllium, magnesium, calcium, strontium, and barium or combinations thereof. The amounts of alkaline earth metal promoters can be used in amounts similar to those used for the additional alkali metal promoters.

The catalyst may also include a promoting amount of a main group element or a mixture of two or more main group elements. Suitable main group elements include any of the elements in Groups IIIA (boron group) to VIIA (halogen group) of the Periodic Table of the Elements. For example, the catalyst can include a promoting amount of sulfur, phosphorus, boron, halogen (e.g., fluorine), gallium, or a combination thereof.

In a preferred embodiment, the catalyst comprises sulfur. Preferably, the catalyst comprises an amount of sulfur cs of 10.0 mmol or less per kg of catalyst, preferably 0.1 to 5.0 mmol per kg of catalyst.

The catalyst may also include a promoting amount of a rare earth metal or a mixture of two or more rare earth metals. The rare earth metals include any of the elements having an atomic number of 57 to 103. Some examples of these elements include lanthanum (La), cerium (Ce), and samarium (Sm). The amount of rare earth metal promoters can be used in amounts similar to those used for the transition metal promoters.

The catalyst comprises an alumina support, on which silver, cesium, rhenium and tungsten are deposited. The alumina support comprises a high proportion of alumina, i.e. Al₂O₃, and in particular alpha-alumina. Specifically, the alumina support comprises at least 50 wt.-% of alpha-alumina, for example at least 70 wt.-%, at least 80 wt.-%, or at least 90 wt.-%, preferably at least 95 wt.-%, most preferably at least 97.5 wt.-% or at least 99 wt.-%, based on the total weight of the support. Besides alumina, the support may comprise other components, for example other refractory oxides such as zirconia or titania.

The support is a low silicon alpha-alumina support. The support comprises an amount of silicon c_{S-Si} of at most 8.9 mmol per kg of support, preferably at most 5.4 mmol per kg of support, more preferably at most 4.0 mmol per kg of support. In one embodiment, the support comprises an amount of amount of silicon c_{S-Si} of 0.001 mmol per kg of support or more, 0.01 mmol per kg of support or more, 0.1 mmol per kg of support or more, or 0.25 mmol per kg of support or more.

The amount of potassium c_{S-K} of the support is typically in the range of at most 3.0 mmol per kg of support, preferably at most 2.5 mmol per kg of support, more preferably at most 2.0 mmol per kg of support, most preferably at most 1.3 mmol per kg of support. In one embodiment, the support comprises an amount of amount of potassium c_{S-Si} of 0.001 mmol per kg of support or more, 0.01 mmol per kg of support or more, 0.1 mmol per kg of support or more, or 0.25 mmol per kg of support or more.

The amount of sodium c_{S-Na} of the support is typically in the range of at most 4.0 mmol per kg of support, preferably 0.67 to 3.0 mmol per kg of support, more preferably 1.0 to 2.0 mmol per kg of support.

In one embodiment, the support comprises
an amount of potassium C_{S-K} of at most 3.0 mmol per kg of support, preferably at most 2.5 mmol per kg of support, more preferably at most 2.0 mmol per kg of support, most preferably at most 1.3 mmol per kg of support, and
an amount of sodium c_{S-Na} of at most 4.0 mmol per kg of support, preferably 0.67 to 3.0 mmol per kg of support, more preferably 1.0 to 2.0 mmol per kg of support.

In a preferred embodiment, the support comprises
an amount of silicon c_{S-Si} of at most 8.9 mmol per kg of support, preferably at most 5.4 mmol per kg of support, more preferably at most 4.0 mmol per kg of support,
an amount of potassium C_{S-K} of at most 3.0 mmol per kg of support, preferably at most 2.5 mmol per kg of support, more preferably at most 2.0 mmol per kg of support, most preferably at most 1.3 mmol per kg of support, and
an amount of sodium c_{S-Na} of at most 4.0 mmol per kg of support, preferably 0.67 to 3.0 mmol per kg of support, more preferably 1.0 to 2.0 mmol per kg of support.

The support may comprise impurities besides sodium and potassium, such as iron, magnesium, calcium, zirconium in an amount of 2 to 200 mmol/kg, based on the total weight of the support.

It is desirable that the silver is relatively uniformly dispersed on the interior and exterior surfaces of the support. High surface area of the support allows for high dispersion of silver. However, increasing the surface area of the support can foster side reactions that may occur on the surface of the support. alpha-Alumina supports having a BET surface area of 3.0 m²/g or less offer a good compromise between silver dispersion and side reactions.

The support preferably has a BET surface area of 0.5 to 3.0 m²/g, more preferably 1.0 to 2.5 m²/g, most preferably 1.2 m²/g to 2.0 m²/g. The BET method is a standard, well-known method and widely used method in surface science for the measurements of surface areas of solids by physical adsorption of gas molecules. The BET surface is determined according to DIN ISO 9277 herein, unless stated otherwise.

The support is a porous support and generally has a total Hg pore volume in the range of 0.4 to 1.2 mL/g, preferably 0.45 to 0.9 mL/g, more preferably 0.5 to 0.8 mL/g, as determined by mercury porosimetry. The catalyst generally has a total Hg pore volume in the range of 0.2 to 1.0 mL/g, preferably 0.3 to 0.8 mL/g, as determined by mercury porosimetry. Mercury porosimetry may be performed using a Micrometrics AutoPore IV 9500 mercury porosimeter (140 degrees contact angle, 485 dynes/cm Hg surface tension, 60000 psia max head pressure). The Hg porosity is determined according to DIN 66133 herein, unless stated otherwise. It is believed that Hg pore volumes in the above ranges allow for a favorable duration of exposure of the obtained ethylene oxide to the catalyst.

The support preferably has a water absorption in the range of 0.35 to 1.2 mL/g (mL of water/gram of support). Preferably, the water absorption of the support is in the range of 0.4 to 1.0 mL/g, most preferably 0.4 to 0.80 mL/g. Water absorption refers to vacuum cold water uptake measured at a vacuum of 80 mbar absolute.

Vacuum cold water uptake is determined by placing about 100 g of support (“initial support weight”) in a rotating flask, covering the support with deionized water, and rotating the rotary evaporator for 5 min at about 30 rpm. Subsequently, a vacuum of 80 mbar is applied for 3 min, the water and the support are transferred into a glass funnel, and the support is kept in the funnel for about 5 min with occasional shaking in order to ensure that adhering water runs down the funnel. The support is weighed (“final support weight”). The water absorption is calculated by subtracting the initial support weight from the final support weight and then dividing this difference by the initial support weight. It is believed that a water absorption in the above ranges allows for a favorable duration of exposure of the obtained ethylene oxide to the catalyst.

The support preferably comprises individual shaped bodies. The size and shape of the individual shaped bodies and thus of the catalyst is selected to allow a suitable packing of the shaped bodies in a reactor tube. The shaped bodies suitable for the catalysts of the invention are preferably used in reactor tubes with a length from 6 to 14 m and an inner diameter from 20 mm to 50 mm. In general, the support is comprised of individual bodies having a maximum extension in the range of 3 to 20 mm, such as 4 to 15 mm, in particular 5 to 12 mm. The maximum extension is understood to mean the longest straight line between two points on the outer circumference of the support.

The shape of the support is not especially limited, and may be in any technically feasible form, depending, e.g., on the extrusion process. For example, the support may be a solid support or a hollow support, such as a hollow cylinder. In another embodiment, the support may be characterized by a multilobe structure. A multilobe structure is meant to denote a cylinder structure which has a plurality of void spaces, e.g., grooves or furrows, running in the cylinder periphery along the cylinder height. Generally, the void spaces are arranged essentially equidistantly around the circumference of the cylinder. Hollow cylinders are characterized by their geometric dimensions, in particular outer diameter×length×internal diameter. The outer diameter is preferably in the range of 5 to 15 mm, preferably 7 to 10 mm. The length is preferably in the range of 5 to 15 mm, preferably 7 to 11 mm. The internal diameter is preferably in the range of 1 to 5 mm, preferably 2 to 4 mm. Specific examples are hollow cylinders having dimensions of outer diameter (mm)×length (mm)×internal diameter (mm) of 5×5×2, 6×6×3, 7×7×3, 8×8×3, 8×8.5×3, 8.5×8.5×3, 9×9×3, and 9×9×3.5.

In another embodiment, the catalyst support may be in a shape such as described in U.S. Pat. No. 9,409,160 B2 wherein the shaped catalyst body has the form of a cylinder with a base, a cylinder surface, a cylinder axis and at least one continuous opening (a passageway that extends from the first face side surface to the second face side surface of the tableted catalyst support) running parallel to the cylinder axis, and the base of the cylinder has at least four lobes.

The catalyst support may also be in a shape such as described in WO 2012/091898 A2, having at least three lobes, a first end, a second end, a wall between the ends and a non-uniform radius of transition at the intersection an end and the wall.

In one embodiment, the catalyst support has more than one passageway extending from the first face side surface to the second face side surface of the tableted catalyst support. Such shapes are known in the art, as described in the following.

For example, U.S. Pat. No. 5,861,353 A describes catalysts and catalyst carriers in the form of cylindrical granules, characterized in that each granule displays at least three through-bores (passageways that extend from the first face side surface to the second face side surface of the tableted catalyst support) having axes which are substantially parallel to each other and to the axes of the granule, and substantially equidistant from each other.

U.S. Pat. No. 9,138,729 B2 describes a shaped catalyst that has an essentially cylindrical body having a longitudinal axis, wherein the cylindrical body has at least two parallel internal holes (passageways that extend from the first face side surface to the second face side surface of the tableted catalyst support) which are essentially parallel to the cylinder axis of the body and go right through the body, and wherein the internal holes have a round or oval cross section.

WO 2020/108872 A1 describes a shaped catalyst body for producing ethylene oxide by gas-phase oxidation of ethylene, comprising silver deposited on a porous refractory support, the shaped catalyst body having a first face side surface, a second face side surface and a circumferential surface, a cylinder structure with n void spaces running in the cylinder periphery along the cylinder height to form an n-lobed structure, wherein n is 2, 3, 4, 5 or 6, n passageways extending from the first face side surface to the second face side surface, each passageway being assigned to one lobe, wherein neighboring passageways are arranged essentially equidistantly to each other, an n-fold rotational symmetry, a shortest distance A between two neighboring passageways in the range of 1.0 to 2.0 mm and a shortest distance B between each passageway and the circumferential surface in the range of 1.1 to 2.0 mm.

In a preferred embodiment, the alpha-alumina support is a tableted alpha-alumina support. In this case, the support may be obtained by forming a precursor material into shaped bodies via tableting, typically in the absence of a liquid, and subsequently subjecting the shaped bodies to heat treatment. Tableting is a process of press agglomeration. A powdered or previously agglomerated bulk material is introduced into a pressing tool having a die between two punches and compacted by uniaxial compression and shaped to give a solid compact. This operation is divided into four parts: metered introduction, compaction (elastic deformation), plastic deformation and ejection. Tableting is carried out, for example, on rotary presses or eccentric presses.

If desired, the upper punch and/or lower punch may comprise projecting pins to form internal passageways. It is also possible to provide the pressing punches with a plurality of movable pins, so that a punch can, for example, be made with four pins to create shaped bodies with four holes (passageways). Typical design features of such tools may be found in, e.g., U.S. Pat. No. 8,865,614 B2.

The tableting process allows for the accurate manufacture of catalyst supports, i.e., the manufacture of a plurality of catalyst supports having relatively little deviations in outer dimensions. Such supports are geometrically nearly identical, which allows for a better calculability of their behavior in reaction processes and a lower pressure loss in, e.g., gas-phase catalysis.

The shape of the tableted catalyst support is not particularly limited, as long as it is accessible by a conventionally known tableting press of the punch-and-die type. The shapes of the tableted catalyst support are generally such that each is composed of a circumferential surface, which corresponds to the internal wall of the die cavity, and a top face side surface and a bottom face side surface, which correspond to the operative heads of the punches. It is also possible that the upper punch and lower punch come together during the tableting process. In this case, no discrete circumferential and face side surfaces are formed. Thus, tableted catalyst supports having an outer shape of, e.g., a sphere or an ellipsoid may be obtained.

For tableting, it is often preferable to make use of lubricants, in particular those discussed above. To improve tableting properties, a pre-granulation and/or sieving step may be used. For pre-granulation, a roll compactor, such as a Chilsonator® from Fitzpatrick, may be used. Further information regarding tableting, in particular with regard to pre-granulation, sieving, lubricants and tools, may be found in WO 2010/000720 A2.

It has been found that highly porous transition aluminas having a low bulk density are useful starting materials for the production of alpha-alumina catalyst supports with beneficial pore structure, in particular transition aluminas having relatively high pore volume and large pore diameters. Such transition aluminas are suitable for shaping via the tableting process to obtain geometrically accurate supports with high total pore volume. Further details regarding useful starting materials and the tableting process itself have been described, e.g., in WO 2021/260185 A1.

Moreover, the tableting technique allows for the use of specific pore-forming materials that are particularly suitable for obtaining an advantageous pore structure while allowing for a catalyst support having high purity. Thus, low concentrations of sodium and potassium in the support may be achieved. The pore-forming materials notably include substances which cannot be used or controlled easily in extrusion processes due to their tendency to lose their structural integrity under extrusion conditions, such as water-soluble, moisture-liable or shear-degradable pore-forming materials. Suitable pore-forming materials include thermally decomposable materials such as ammonium bicarbonate, ammonium carbonate, ammonium carbamate, ammonium nitrate, urea, malonic acid and oxalic acid, in particular malonic acid and ammonium bicarbonate; and organic polymers such as microcrystalline cellulose and cellulose-fiber granule, such as agglomerated spray-dried cellulose fibers (cellulose pulp granule), in particular ammonium bicarbonate.

In one embodiment, the support is in the shape of a solid extrudate, such as pellets or cylinders, or a hollow extrudate, such as a hollow cylinder. In a preferred embodiment, the shaped bodies are formed by extrusion, e.g., micro-extrusion, of a precursor material.

In this case, the precursor material suitably comprises a liquid, in particular water, so as to form a malleable precursor material. The precursor material typically comprises alpha-alumina, an alumina hydrate and/or a transition alumina. Further details regarding useful starting materials and the extrusion process itself have been described, e.g., in WO 2021/260182 A1.

In a preferred embodiment, extrusion comprises charging at least one solid component into a mixing device before the liquid is added. Preferably, a mix-muller (H-roller) or a horizontal mixer such as a Ploughshare® mixer (from Gebrüder Lödige Maschinenbau) is used for mixing. The forming of an extrudable paste of the precursor material can be monitored and controlled based on data reflecting power consumption of the mixing device.

The precursor material is typically extruded through a die. The cross-section of the die opening is adapted according to the desired geometry of the shaped body.

The extrusion die may comprise a matrix and mandrels, wherein the matrix essentially determines the circumferential shape of the shaped bodies and the mandrels essentially determine the form, size and position of passageways, if present. Suitable extrusion dies are described in, e.g., WO 2019/219892 A1.

The geometry of the shape of the shaped bodies is defined by the geometry of the extrusion apparatus through which the precursor material is extruded. Generally, the geometry of the shape of the extrudate differs slightly from the geometry of the extrusion apparatus, while essentially having the geometric properties described above. Absolute sizes of the shape are in general slightly lower than the sizes of the extrudate, due to high temperatures required to form alpha alumina and shrinkage upon cooling of the extrudate. The extent of the shrinkage depends on the temperatures applied during calcination and the components of the shaped bodies. Therefore, the size of the extrusion dies should be routinely adjusted in a way to account for the extrudate shrinkage during the subsequent calcination.

When the shaped body comprises multiple passageways, the axes of the passageways typically run parallel. However, the shaped bodies may be slightly bent or twisted along their z axis (height). The shape of the cross-section of the passageways may be slightly different from the envisioned perfect geometrical shapes described above. When a large amount of shaped bodies is obtained, single passageways of a small number of the shaped catalyst bodies may be closed. Usually the face sides of the shaped catalyst bodies in the xy plane are more or less uneven, rather than smooth, due to the production process. The height of the shaped bodies (length of the shaped bodies in the z direction) is usually not exactly the same for all of the shaped bodies, but rather constitutes a distribution with an average height as its arithmetic mean.

The extrudate is preferably cut into the desired length while still wet. Preferably, the extrudate is cut at an angle essentially perpendicular to its circumferential surface. In order to reduce undesirable deviations from the geometry of the extrusion apparatus, the extrudate may alternatively be cut at a slanted angle of up to 30°, such as 100 or 20°, with regard to the angle perpendicular to the circumferential surface of the extrudate.

Aberrations from the geometry as incurred in the extrusion process and/or the further processing of the extrudate, e.g. a cutting step, may generally also be present in the alpha-alumina catalyst support. The skilled person understands that perfect geometrical forms are fundamentally unobtainable due to the imprecision which is inherent to all production processes to some degree.

In another embodiment, the precursor material is formed into shaped bodies using a micro-extrusion process such as the one described in WO 2019/072597 A1.

In another embodiment, the precursor material is formed into shaped bodies using a gel casting process such as the one described in WO 2020/053563 A1.

The invention moreover relates to a process for preparing an epoxidation catalyst as described above, comprising
i) impregnating an alumina support as described above with a silver impregnation solution; and
ii) subjecting the impregnated support to a heat treatment;
wherein steps i) and ii) are optionally repeated, and at least one silver impregnation solution comprises rhenium, tungsten, cesium, potassium, and optionally sodium.

It is understood that all embodiments of the catalyst and support also apply to the process for preparing the catalyst, where applicable.

In order to obtain a shaped catalyst body having high silver contents, steps i) and ii) can be repeated several times. In that case it is understood that the intermediate product obtained after the first (or subsequent up to the last but one) impregnation/heat treatment cycle comprises a part of the total amount of target Ag and/or promoter concentrations.

The intermediate product is then again impregnated with the silver impregnation solution and calcined to yield the target Ag and/or promoter concentrations. It is also possible to establish the desired composition of the catalyst by applying only one impregnation.

Any silver impregnation solution suitable for impregnating a refractory support known in the art can be used. Silver impregnation solutions typically contain a silver carboxylate, such as silver oxalate, or a combination of a silver carboxylate and oxalic acid, in the presence of an aminic complexing agent like a C₁-C₁₀-alkylenediamine, in particular ethylenediamine. Suitable impregnation solutions are described in EP 0 716 884 A2, EP 1 115 486 A1, EP 1 613 428 A1, U.S. Pat. No. 4,731,350 A, WO 2004/094055 A2, WO 2009/029419 A1, WO 2015/095508 A1, U.S. Pat. Nos. 4,356,312 A, 5,187,140 A, 4,908,343 A, 5,504,053 A, WO 2014/105770 A1 and WO 2019/154863. Cesium may suitably be provided as cesium hydroxide. Rhenium and tungsten may suitably be provided as an oxyanion, for example, as a perrhenate or tungstate in salt or acid form.

At least one silver impregnation solution comprises rhenium, tungsten, cesium, potassium, and optionally sodium. It is especially preferred that at least the silver impregnation solution employed in the final impregnation step comprises rhenium, tungsten, cesium and potassium.

During heat treatment, liquid components of the silver impregnation solution evaporate, causing a silver compound comprising silver ions to precipitate from the solution and be deposited onto the porous support. At least part of the deposited silver ions is subsequently converted to metallic silver upon further heating. Preferably, at least 70 mol-% of the silver compounds, preferably at least 90 mol-%, more preferably at least 95 mol-% and most preferably at least 99.5 mol-% or at least 99.9 mol-%, i.e. essentially all of the silver ions, based on the total molar amount of silver in the impregnated porous alpha-alumina support, respectively. The amount of the silver ions converted to metallic silver can for example be determined via X-ray diffraction (XRD) patterns.

The heat treatment may also be referred to as a calcination process. Any calcination processes known in the art for this purpose can be used. Suitable examples of calcination processes are described in U.S. Pat. Nos. 5,504,052 A, 5,646,087 A, 7,553,795 A, 8,378,129 A, 8,546,297 A, US 2014/0187417 A1, EP 1 893 331 A1 or WO 2012/140614 A1. Heat treatment can be carried out in a pass-through mode or with at least partial recycling of the calcination gas.

Heat treatment is usually carried out in a furnace. The type of furnace is not especially limited. For example, stationary circulating air furnaces, revolving cylindrical furnaces or conveyor furnaces may be used. In one embodiment, heat treatment constitutes directing a heated gas stream over the impregnated bodies. The duration of the heat treatment is generally in the range of 5 min to 20 h, preferably 5 min to 30 min.

The temperature of the heat treatment is generally in the range of 200 to 800 °C, preferably 210 to 650 °C, more preferably 220 to 500 °C, most preferably 220 to 350 °C. Preferably, the heating rate in the temperature range of 40 to 200 °C is at least 20 K/min, more preferably at least 25 K/min, such as at least 30 K/min. A high heating rate may be achieved by directing a heated gas over the impregnated refractory support or the impregnated intermediate catalyst at a high gas flow.

A suitable flow rate for the gas may be in the range of, e.g., 1 to 1,000 Nm³/h, 10 to 1,000 Nm³/h, 15 to 500 Nm³/h or 20 to 300 Nm³/h per kg of impregnated bodies. In a continuous process, the term “kg of impregnated bodies” is understood to mean the amount of impregnated bodies (in kg/h) multiplied by the time (in hours) that the gas stream is directed over the impregnated bodies. It has been found that when the gas stream is directed over higher amounts of impregnated bodies, e.g., 15 to 150 kg of impregnated bodies, the flow rate may be chosen in the lower part of the above-described ranges, while achieving the desired effect.

Determining the temperature of the heated impregnated bodies directly may pose practical difficulties. Hence, when a heated gas is directed over the impregnated bodies during heat treatment, the temperature of the heated impregnated bodies is considered to be the temperature of the gas immediately after the gas has passed over the impregnated bodies. In a practical embodiment, the impregnated bodies are placed on a suitable surface, such as a wire mesh or perforated calcination belt, and the temperature of the gas is measured by one or more thermocouples positioned adjacent to the opposite side of the impregnated bodies which first comes into contact with the gas. The thermocouples are suitably placed close to the impregnated bodies, e.g., at a distance of 1 to 30 mm, such as 1 to 3 mm or 15 to 20 mm from the impregnated bodies.

The use of a plurality of thermocouples can improve the accuracy of the temperature measurement. Where several thermocouples are used, these may be evenly spaced across the area on which the impregnated bodies rest on the wire mesh, or the breadth of the perforated calcination belt. The average value is considered to be the temperature of the gas immediately after the gas has passed over the impregnated bodies. To heat the impregnated bodies to the temperatures as described above, the gas typically has a temperature of 220 to 800 °C, more preferably 230 to 550 °C, most preferably 240 to 350 °C.

Preferably, heating takes place in a step-wise manner. In step-wise heating, the impregnated bodies are placed on a moving belt that moves through a furnace with multiple heating zones, e.g., 2 to 8 or 2 to 5 heating zones. Heat treatment is preferably performed in an inert atmosphere, such as nitrogen, helium, or mixtures thereof, in particular in nitrogen.

Further provided is a process for producing ethylene oxide by gas-phase oxidation of ethylene, comprising reacting ethylene and oxygen in the presence of an epoxidation catalyst as described above.

The epoxidation can be carried out by all processes known to those skilled in the art. It is possible to use all reactors which can be used in the ethylene oxide production processes of the prior art; for example externally cooled shell-and-tube reactors (cf. Ullmann's Encyclopedia of Industrial Chemistry, 5th edition, vol. A-10, pp. 117-135, 123-125, VCH-Verlagsgesellschaft, Weinheim 1987) or reactors having a loose catalyst bed and cooling tubes, for example the reactors described in DE 34 14 717 A1, EP 0 082 609 A1 and EP 0 339 748 A2.

The epoxidation is preferably carried out in at least one tube reactor, preferably in a shell-and-tube reactor. On a commercial scale, ethylene epoxidation is preferably carried out in a multi-tube reactor that contains several thousand tubes. The catalyst is filled into the tubes, which are placed in a shell that is filled with a coolant. In commercial applications, the internal tube diameter is typically in the range of 20 to 40 mm (see, e.g., U.S. Pat. No. 4,921,681 A) or more than 40 mm (see, e.g., WO 2006/102189 A1).

To prepare ethylene oxide from ethylene and oxygen, it is possible to carry out the reaction under conventional reaction conditions as described, e.g., in DE 25 21 906 A, EP 0 014 457 A2, DE 23 00 512 A1, EP 0 172 565 A2, DE 24 54 972 A1, EP 0 357 293 A1, EP 0 266 015 A1, EP 0 085 237 A1, EP 0 082 609 A1 and EP 0 339 748 A2. Inert gases such as nitrogen or gases which are inert under the reaction conditions, e.g. steam, methane, and also optionally reaction moderators, for example halogenated hydrocarbons such as ethyl chloride, vinyl chloride or 1,2-dichloroethane can additionally be mixed into the reaction gas comprising ethylene and molecular oxygen.

The oxygen content of the reaction gas is advantageously in a range in which no explosive gas mixtures are present. A suitable composition of the reaction gas for preparing ethylene oxide can, for example, comprise an amount of ethylene in the range from 10 to 80% by volume, preferably from 20 to 60% by volume, more preferably from 25 to 50% by volume and particularly preferably in the range from 25 to 40% by volume, based on the total volume of the reaction gas. The oxygen content of the reaction gas is advantageously in the range of not more than 10% by volume, preferably not more than 9% by volume, more preferably not more than 8% by volume and very particularly preferably not more than 7.5% by volume, based on the total volume of the reaction gas. The reaction gas preferably comprises a chlorine-comprising reaction moderator such as ethyl chloride, vinyl chloride or 1,2-dichloroethane in an amount of from 0 to 15 ppm by weight, preferably in an amount of from 0.1 to 8 ppm by weight, based on the total weight of the reaction gas. The remainder of the reaction gas generally comprises hydrocarbons such as methane and also inert gases such as nitrogen. In addition, other materials such as steam, carbon dioxide or noble gases can also be comprised in the reaction gas.

The concentration of carbon dioxide in the feed (i.e. the gas mixture fed to the reactor) typically depends on the catalyst selectivity and the efficiency of the carbon dioxide removal equipment. Carbon dioxide concentration in the feed is preferably at most 3 vol.-%, more preferably less than 2 vol.-%, most preferably less than 1 vol.-%, relative to the total volume of the feed. An example of carbon dioxide removal equipment is provided in U.S. Pat. No. 6,452,027 B1.

The above-described constituents of the reaction mixture may optionally each have small amounts of impurities. Ethylene can, for example, be used in any degree of purity suitable for the gas-phase oxidation according to the invention. Suitable degrees of purity include, but are not limited to, “polymer-grade” ethylene, which typically has a purity of at least 99%, and “chemical-grade” ethylene which typically has a purity of less than 95%. The impurities typically comprise, in particular, ethane, propane and/or propene.

The reaction or oxidation of ethylene to ethylene oxide is usually carried out at elevated catalyst temperatures. Preference is given to catalyst temperatures in the range of 150 to 350 °C, more preferably 180 to 300 °C, particularly preferably 190 to 280 °C and especially preferably 200 to 280 °C. The present invention therefore also provides a process as described above in which the oxidation is carried out at a catalyst temperature in the range 180 to 300 °C, preferably 200 to 280 °C. Catalyst temperature can be determined by thermocouples located inside the catalyst bed. As used herein, the catalyst temperature or the temperature of the catalyst bed is deemed to be the weight average temperature of the catalyst particles.

The reaction according to the invention (oxidation) is preferably carried out at pressures in the range of 5 to 30 bar. All pressures herein are absolute pressures, unless noted otherwise. The oxidation is more preferably carried out at a pressure in the range of 5 to 25 bar, such as 10 bar to 24 bar and in particular 14 bar to 23 bar. The present invention therefore also provides a process as described above in which the oxidation is carried out at a pressure in the range of 14 bar to 23 bar.

The physical characteristics of the shaped catalyst body, especially the BET surface area and the pore size distribution, may have a significant positive impact on the catalyst selectivity. This effect is especially pronounced when the catalyst is operated at very high work rates, i.e., high levels of olefin oxide production.

The process according to the invention is preferably carried out under conditions conducive to obtain a reaction mixture containing at least 2.3 vol.-% of ethylene oxide.

In other words, the ethylene oxide outlet concentration (ethylene oxide concentration at the reactor outlet) is preferably at least 2.3 vol.-%. The ethylene oxide outlet concentration is more preferably in the range of 2.5 to 4.0 vol.-%, most preferably in the range of 2.7 to 3.5 vol.-%.

The oxidation is preferably carried out in a continuous process. If the reaction is carried out continuously, the GHSV (gas hourly space velocity) is, depending on the type of reactor chosen, for example on the size/cross-sectional area of the reactor, the shape and size of the catalyst, preferably in the range from 800 to 10,000/h, preferably in the range from 2,000 to 8,000/h, more preferably in the range from 2,500 to 6,000/h, most preferably in the range from 4,500 to 5,500/h, where the values indicated are based on the volume of the catalyst.

According to a further embodiment, the present invention is also directed to a process for preparing ethylene oxide (EO) by gas-phase oxidation of ethylene by means of oxygen as disclosed above, wherein the EO-space-time-yield measured is greater than 180 kg_{EO}/(m³_{cal}h), preferably to an EO-space-time-yield of greater than 200 kg_{EO}/(m³_{cal}h), such as greater than 250 kg_{EO}/(m³_{cal}h), greater than 280 kg_{EO}/(m³_{cal}h), or greater than 300 kg_{EO}/(m³_{cal}h). Preferably the EO-space-time-yield measured is less than 500 kg_{EO}/(m³_{cal}h), more preferably the EO-space-time-yield is less than 350 kg_{EO}/(m³_{cal}h).

The preparation of ethylene oxide from ethylene and oxygen can advantageously be carried out in a recycle process. After each pass, the newly formed ethylene oxide and the by-products formed in the reaction are removed from the product gas stream. The remaining gas stream is supplemented with the required amounts of ethylene, oxygen and reaction moderators and reintroduced into the reactor. The separation of the ethylene oxide from the product gas stream and its work-up can be carried out by customary methods of the prior art (cf. Ullmann's Encyclopedia of Industrial Chemistry, 5th edition, vol. A-10, pp. 117-135,123-125, VCH-Verlagsgesellschaft, Weinheim 1987).

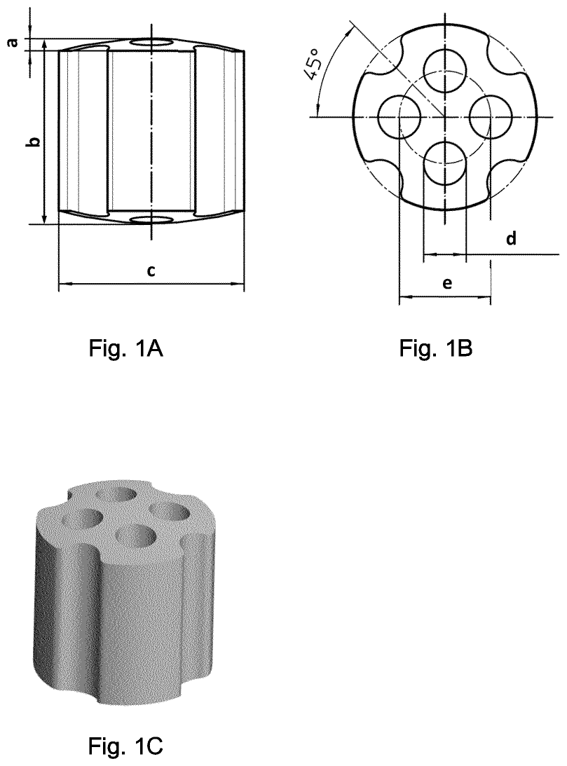

FIG. 1A to 1C show the shape of the supports used in the preparation of the catalysts of the examples. FIGS. 1A and 1C show side views, FIG. 1 B shows a top view. The support has domed face side surfaces having dome heights a, length b, outer diameter c, passageway diameters d and a distance between the centers of the passageways e.

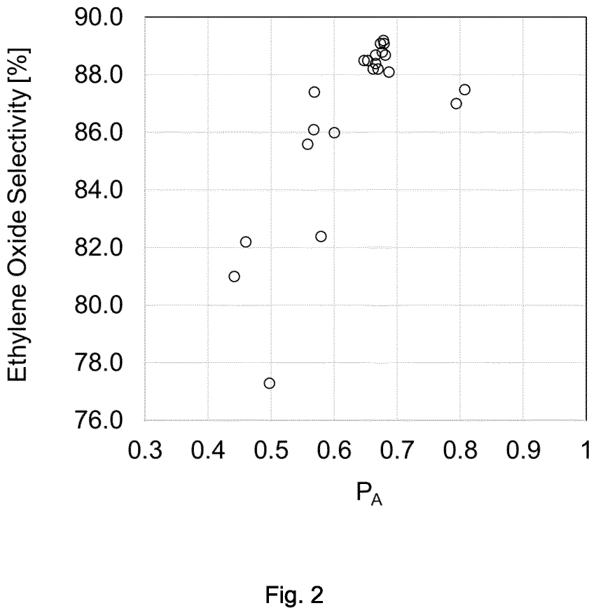

FIG. 2 shows the P_{A} values of different catalysts and their ethylene oxide selectivity [%] after 11 days on stream.

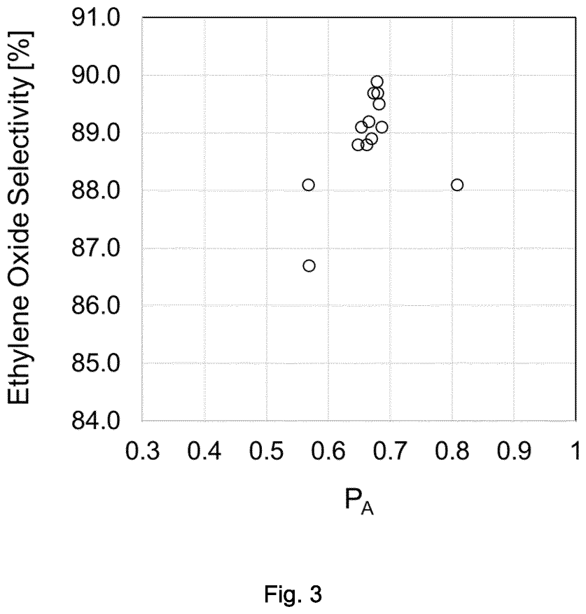

FIG. 3 shows the P_{A} values of different catalysts and their ethylene oxide selectivity [%] after 25 days on stream.

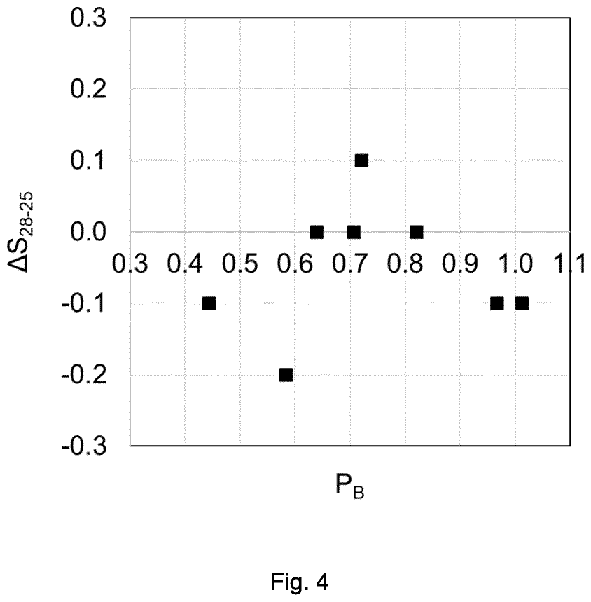

FIG. 4 shows the P_{B} values of different catalysts and their ΔS₂₈₋₂₅ values, i.e., the difference in selectivity after 28 days and after 25 days on stream.

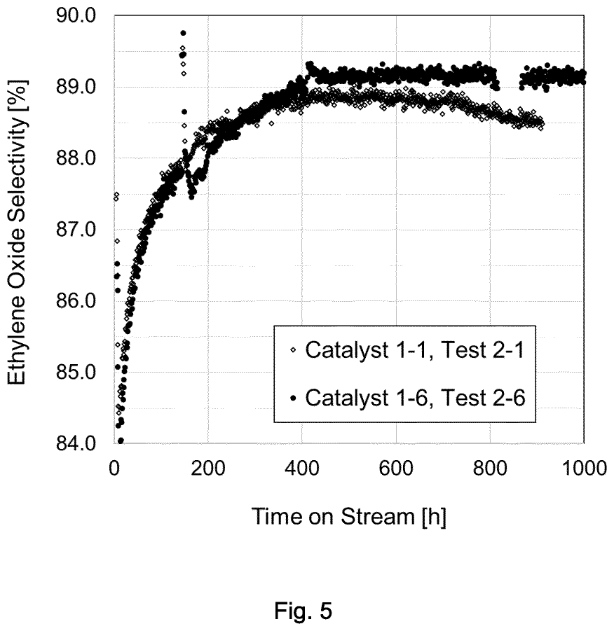

FIG. 5 shows the ethylene oxide selectivity [%] of catalysts 1-1 and 1-6 over time on stream.

The invention will be described in more detail by the subsequent examples.

### Method 1: Analysis of the Total Amount of Ca-, Mg-, Si-, Fe-, K-, Na-, and Ti-Contents in Alpha-Alumina Supports

### 1A. Sample Preparation

Two aliquots of a sample (0.1 to 0.2 g) were weighed into microwave digestion vessels. Then, 10.5 mL of an acid mixture (6.5 mL of phosphoric acid (80 to 85%), 3.5 mL of sulfuric acid (96%) and 0.5 mL of nitric acid (65%)) were added. The microwave digestion vessels were placed in a microwave digestion system, heated up to 230 °C and held at this temperature for 45 min.

After cooling down, the samples were transferred into a volumetric flask and filled up to a volume of 50 mL with de-ionized water. Blank samples s were prepared by the same procedure.

### 1 B. Measurement

The resulting digested solution was analyzed by inductively coupled plasma optical emission spectrometry (ICP-OES) using internal standard (Sc) and blank subtraction.
Instrument: Thermo iCAP Pro XP Duo
Wavelength: K 766.490 nm
Dilution: Direct measurement (no dilution)
Calibration: External
Integration time: 10 s/replicate
Replicates: 3
Plasma power: 1.20 kW
Pump speed: 10 rpm
Nebulizer: Conical 1 mL
Nebulizer gas: Ar, 0.70 L/min

The reported results were calculated from the mean of the two prepared replicates for each sample.

### Method 2: Mercury Porosimetry

Mercury porosimetry was performed using a Micrometrics AutoPore IV 9500 mercury porosimeter (140 degrees contact angle, 485 dynes/cm Hg surface tension, 60,000 psia max head pressure). Mercury porosity was determined in accordance with DIN 66133.

### Method 3: Nitrogen Sorption

Nitrogen sorption measurements were performed using a Micrometrics ASAP 2420. Nitrogen porosity was determined in accordance with DIN 66134. The sample was degassed at 200 °C for 16 h under vacuum prior to the measurement.

### Method 4: BET Surface Area

The BET surface area was determined in accordance with DIN ISO 9277.

### Method 5: Water Absorption

Water absorption refers to vacuum cold water uptake. Vacuum cold water uptake is determined by placing about 100 g of support (“initial support weight”) in a rotating flask, covering the support with deionized water, and rotating the rotary evaporator for 5 min at about 30 rpm. Subsequently, a vacuum of 80 mbar is applied for 3 min, the water and the support are transferred into a glass funnel, and the support is kept in the funnel for about 5 min with occasional shaking in order to ensure that adhering water runs down the funnel.

The support is weighed (“final support weight”). The water absorption is calculated by subtracting the initial support weight from the final support weight and then dividing this difference by the initial support weight.

### EXAMPLES

### Example 1—Preparation of Supports

Alumina raw materials, as specified in Table 1 (transition aluminas and alumina hydrates, obtained from Sasol), and Vaseline-coated ammonium bicarbonate (ABC-O, BASF) as pore forming material were mixed with Kolliwax® HCO (hydrogenated castor oil waxy mass from BASF) and Timrex® T44 (graphite from TimCal Graphite & Carbon) as processing aids to obtain a free-flowing powder mixture. Vaseline-coated ammonium bicarbonate was prepared by mixing 99 wt.-% of ammonium bicarbonate with 1 wt.-% of Vaseline® (Unilever) in a plowshare mixer (e.g. L5, Gebrüder Lödige Maschinenbau) at 175 rpm for 20 min. The weight ratio of all components in the formulations are shown in Table 1.

TABLE 1

| | Transition | Alumina | Pore-Forming | Processing |
|---|---|---|---|---|
| Support | Alumina | Hydrate | Material | Aid |
| A | Puralox TH | Pural TH 200 | Vaseline-coated | Kolliwax HCO |
| | 200/70 | 20 g | (NH₄)HCO₃ | 5 g |
| | 80 g | | 66 g | Timrex T44 |
| | | | | 5 g |
| B to D | Puralox TH | Pural TH 200 | Vaseline-coated | Kolliwax HCO |
| | 200/70 | 20 g | (NH₄)HCO₃ | 5 g |
| | 80 g | | 76 g | Timrex T44 |
| | | | | 5 g |

The powder mixtures were each subjected to tableting in a rotary tableting machine (Kilian E150 Plus, Romaco) equipped with a tetralobe punch having four holes with an outer diameter of about 16.5 mm and a hole diameter of about 3.8 mm. The tablets were produced at a pre-compaction pressure in the range of 0.5 to 0.9 kN, a main compaction pressure in the range of 7 to 10 kN, and a rotation speed of 8 to 11 rpm. The average height of the obtained green tablets was 12.5 mm.

The green tablets were thermally treated in a muffle furnace. The furnace temperature was ramped up to 500 °C at a heating rate of 2 °C/min, held at 500 °C for 30 min, then ramped up to the target temperature with a heating rate of 2 °C/min and held at the target temperature for 4 h. Heat treatment was performed under lean air with 5 vol.-% oxygen.

The final shape of tetralobe tableted supports is shown in FIGS. 1A to 1C, depicting side views and a top view. The support has domed face side surfaces having dome heights a of 0.88 mm each, a length b of 9.6 mm, an outer diameter c of 13.2 mm, passageway diameters d of 3.0 mm each and a distance between the centers of the passageways e of 6.6 mm each.

In order to reach the target BET surface area, each support was calcined at the specific temperature indicated in Table 2. Table 2 shows the physical properties of supports A to D. Table 3 shows the Ca-, Mg-, Si-, Fe-, K-, Na-, and Ti-contents of supports A to D.

TABLE 2

| | | | | | | | | | Peak | |
|---|---|---|---|---|---|---|---|---|---|---|
| | | | | | | | | | Pore Diameter | |
| | | BET | Total | | | | | | in Pore Size | |
| | Calcination | Surface | Pore | Pore Volume Contained in Pores [mL/g] * | | | | | Distribution | |
| | Temp. | Area | Volume | (Proportion of the Total Pore Volume [%]) | | | | | [μm]* | |
| Support | [°C] | [m²/g] | [mL/g] | <0.2 μm | <0.3 μm | 0.1-1 μm | 1-10 μm | >10 μm | 1^{st} | 2^{nd} |
| A | 1450 | 1.95 | 0.55 | 0 | 0.01 | 0.27 | 0.20 | 0.07 | 0.46 | 7 |
| | | | | (0) | (1.8) | (49.0) | (36.5) | (12.7) | | |
| B | 1480 | 2.01 | 0.57 | 0 | 0.01 | 0.24 | 0.15 | 0.18 | 0.46 | 14 |
| | | | | (0) | (1.8) | (42.1) | (26.3) | (31.6) | | |
| C | 1500 | 1.67 | 0.53 | 0 | 0 | 0.22 | 0.15 | 0.16 | 0.52 | 13 |
| | | | | (0) | (0) | (41.5) | (28.3) | (30.2) | | |
| D | 1525 | 1.40 | 0.49 | 0 | 0 | 0.19 | 0.15 | 0.15 | 0.61 | 12 |
| | | | | (0) | (0) | (38.8) | (30.6) | (30.6) | | |

*determined by mercury porosimetry

TABLE 3

| Support | | Ca | Mg | K | Na | Si | Fe | Ti |
|---|---|---|---|---|---|---|---|---|
| A | ppmw | <100 | <100 | <10 | 40 | 100 | 100 | <100 |
| | mmol/kg | <2.50 | <4.11 | <0.26 | 1.74 | 3.56 | 1.79 | <2.09 |
| B to D | ppmw | <100 | <100 | <10 | 45 | <100 | <100 | <100 |
| | mmol/kg | <2.50 | <4.11 | <0.26 | 1.96 | <3.56 | <1.79 | <2.09 |

### Example 2—Preparation of Shaped Catalyst Bodies

Shaped catalyst bodies according to Table 6 below were prepared by impregnating support A to D with a silver impregnation solution.

### 2.1 Production of the Silver Complex Solution

689 kg of an aqueous ethylenediamine solution with an ethylenediamine content of 58 wt.-% was pumped into a stirring reactor 1. Subsequently, the 58 wt.-% ethylenediamine solution was diluted under stirring with 140 kg of de-ionized water. Next, 24.5 kg of 0.9 wt.-% aqueous KOH solution were added to form an aqueous KOH/ethylenediamine solution. Then, 300 kg of oxalic acid dihydrate (purity>99.6%) were added into the stirring reactor 1 stepwise (total addition time about 1 h). During the addition of oxalic acid dihydrate, the temperature was controlled in the range of 18 to 38 °C. After the addition of the last portion of oxalic acid dihydrate, the reaction mixture was stirred for the next 30 minutes at a temperature in the range of 25 to 30 °C to form an aqueous oxalic acid-ethylenediamine solution.

Next, 1052 kg of the resulting aqueous oxalic acid-ethylenediamine solution was transferred from the stirring reactor 1 to a stirring reactor 2. Then, 500 kg silver oxide powder (Ag₂O-content>99.90%) were added into the stirring reactor 2 stepwise (total addition time about 1 h). During the addition of silver oxide, the temperature was controlled in the range of 18 to 38 °C. After the addition of the last portion of silver oxide, the reaction mixture was stirred for the next 60 minutes at a temperature in the range of 25 to 35 °C to form an aqueous silver complex solution or suspension. The solution or suspension was passed through a filtration unit to remove undissolved solid. The resulting silver complex solution had a density 1.547 g/ml and a silver content of 29.9 wt.-% and a potassium content of 90 ppmw.

### 2.2. Preparation of Ag-Containing Intermediates

145.6 g of support A were placed into a 2 L glass flask. The flask was attached to a rotary evaporator, which was set under a vacuum pressure of 80 mbar. The rotary evaporator system was set in rotation of 30 rpm. 108.0 g of silver complex solution prepared according to step 2.1 were added onto support A over 15 min under a vacuum pressure of 80 mbar. After addition of the silver complex solution, the rotary evaporator system was continued to rotate under vacuum for a further 15 min. The impregnated support was then left in the apparatus at room temperature (approximately 25 °C) and atmospheric pressure for 1 h and mixed gently every 15 min.

The impregnated material was placed on a net forming 1 to 2 layers (about 100 to 200 g per calcination run). The net was subjected to 23 Nm³/h of nitrogen flow, wherein the gas flow was pre-heated to a temperature of 305 °C. The impregnated materials were heated up to a temperature of 290 °C at a heating rate of about 30 K/min and then maintained at 290 °C for 8 min to yield Ag-containing intermediate product I-1 according to Table 4. The temperature was measured by placing three thermocouples at 1 mm below the calcination net. Subsequently, the catalysts were cooled to ambient temperature by removing the intermediate catalyst bodies from the net using an industrial vacuum cleaner.

Ag-containing intermediate products I-2 to I-5 were prepared analogously using supports A to D, as indicated in Table 4, by adjusting the weight ratios of the used supports and the Ag-containing complex solutions.

TABLE 4
(Ag-contents are calculated values based on the amounts of used
supports and Ag-complex solution and reported in percent by weight,
relative to the total weight of the catalyst)

| Intermediate Product | Support | Ag [wt.-%] |
|---|---|---|
| I-1 | A | 18.15 |
| I-2 | A | 18.75 |
| I-3 | B | 18.75 |
| I-4 | C | 18.75 |
| I-5 | D | 17.71 |

### 2.3. Preparation of Final Catalysts

An amount of Ag-containing intermediate product as indicated in Table 5 was placed into a 2 L glass flask. The flask was attached to a rotary evaporator, which was set under vacuum pressure of 80 mbar. The rotary evaporator system was set in rotation of 30 rpm. An amount of the silver complex solution listed in Table 5 prepared according to step 2.1 was mixed with amounts of promoter solution I, promoter solution II and promoter solution Ill as listed in Table 5.

Promoter solution I was obtained by dissolving lithium nitrate (FMC, 99.3%) and ammonium sulfate (Merck, 99.4%) in deionized (DI) water to achieve a target Li content of 2.85 wt.-% and a target S content of 0.182 wt.-%.

Promoter solution II was obtained by dissolving tungstic acid (HC Starck, 99.99%) in DI water and a mixture of cesium hydroxide in water (HC Starck, 50.42%), potassium hydroxide in water (BASF, 2.0%) and sodium hydroxide in water (BASF, 2.0%) to achieve target Cs, W, K and Na contents listed in Table 5.

Promoter solution Ill was obtained by dissolving ammonium perrhenate (Buss & Buss Spezialmetalle GmbH, 99.9%) in a 29 wt.-% aqueous solution of ethylene diamine to achieve a target Re content of 10.0 wt.-%.

The combined impregnation solution containing silver complex solution, promoter solutions I, II, and III and an amount of DI water as listed in Table 5 was stirred for 5 min. The combined impregnation solution was added onto an amount of the silver-containing intermediate product prepared according to step 2.2 listed in Table 4 over 15 min under a vacuum pressure of 80 mbar. After addition of the combined impregnation solution, the rotary evaporator system was continued to rotate under vacuum for another 15 min. The impregnated support was then left in the apparatus at room temperature (about 25 °C) and atmospheric pressure for 1 h and gently mixed every 15 min.

TABLE 5

| Catalyst | 1-1 | 1-2 | 1-3 | 1-4 | 1-5 | 1-6 |
|---|---|---|---|---|---|---|
| Ag-containing Intermediate | I-1 | I-2 | I-2 | I-2 | I-2 | I-3 |
| Amount of Ag-containing | 172.2 | 175.6 | 175.4 | 175.9 | 176.5 | 175.8 |
| Intermediate [g] | | | | | | |
| Amount of Ag-complex | 80.346 | 84.845 | 84.761 | 85.027 | 85.310 | 84.940 |
| solution [g] | | | | | | |
| Amount of promoter | 3.248 | 3.326 | 3.323 | 3.333 | 3.344 | 3.329 |
| solution I [g] | | | | | | |
| Amount of promoter | 4.135 | 3.729 | 4.568 | 4.582 | 3.750 | 4.241 |
| solution II [g] | | | | | | |
| Cs-/W-/K-/Na-content in | 5.0/3.0/ | 6.49/3.0/ | 5.29/3.0/ | 5.07/3.0/ | 6.49/3.0/ | 4.52/3.0/ |
| promoter solution II [wt.-%] | 0.0/0.0 | 0.63/0.22 | 0.52/0.18 | 0.52/0.18 | 0.35/0.11 | 0.2/0.05 |
| Amount of promoter | 2.599 | 2.359 | 2.357 | 2.971 | 2.981 | 2.665 |
| solution III [g] | | | | | | |
| Amount of DI H₂O [g] | 0.0 | 1.416 | 0.560 | 0.0 | 0.792 | 2.716 |
| Catalyst | 1-7 | 1-8 | 1-9 | 1-10 | 1-11 | 1-12 |
| Ag-containing Intermediate | I-3 | I-3 | I-3 | I-3 | I-3 | I-3 |
| Amount of Ag-containing | 175.9 | 176.3 | 176.2 | 176.4 | 174.8 | 174.8 |
| Intermediate [g] | | | | | | |
| Amount of Ag-complex | 85.012 | 85.157 | 85.128 | 85.234 | 84.439 | 84.439 |
| solution [g] | | | | | | |
| Amount of promoter | 3.332 | 3.338 | 3.337 | 3.341 | 3.310 | 3.310 |
| solution I [g] | | | | | | |
| Amount of promoter | 4.243 | 4.249 | 4.249 | 4.253 | 4.213 | 4.213 |
| solution II [g] | | | | | | |
| Cs-/W-/K-/Na-content in | 5.0/3.0/ | 4.05/3.0/ | 5.0/3.0/ | 4.05/3.0/ | 4.05/3.0/ | 4.05/3.0/ |
| promoter solution II [wt.-%] | 0.2/0.05 | 0.2/0.05 | 0.2/0.05 | 0.2/0.05 | 0.55/0.0 | 0.32/0.0 |
| Amount of promoter | 2.970 | 2.368 | 2.367 | 2.978 | 2.489 | 2.489 |
| solution III [g] | | | | | | |
| Amount of DI H₂O [g] | 2.399 | 3.052 | 2.680 | 2.071 | 4.803 | 4.803 |
| Catalyst | 1-13 | 1-14 | 1-15 | 1-16 | 1-17 | 1-18 |
| Ag-containing Intermediate | I-3 | I-4 | I-4 | I-4 | I-4 | I-5 |
| Amount of Ag-containing | 175.6 | 176.5 | 177.1 | 176.0 | 176.1 | 173.8 |
| Intermediate [g] | | | | | | |
| Amount of Ag-complex | 84.867 | 85.278 | 85.568 | 85.061 | 85.060 | 77.318 |
| solution [g] | | | | | | |
| Amount of promoter | 3.327 | 3.343 | 3.354 | 3.334 | 3.334 | 3.259 |
| solution I [g] | | | | | | |
| Amount of promoter | 4.236 | 4.258 | 4.273 | 4.246 | 4.244 | 4.151 |
| solution II [g] | | | | | | |
| Cs-/W-/K-/Na-content in | 5.0/3.0/ | 4.52/3.0/ | 4.52/3.0/ | 5.0/3.0/ | 4.05/3.0/ | 4.52/3.0/ |
| promoter solution II [wt.-%] | 0.9/0.0 | 0.2/0.0 | 0.2/0.05 | 0.2/0.0 | 0.2/0.0 | 0.2/0.0 |
| Amount of promoter | 2.965 | 2.676 | 2.685 | 2.972 | 2.365 | 2.608 |
| solution III [g] | | | | | | |
| Amount of DI H₂O [g] | 4.326 | 0.255 | 0.256 | 0.0 | 0.583 | 0.310 |

| Catalyst | 1-19 | 1-20 | 1-21 | 1-22 |
|---|---|---|---|---|
| Ag-containing Intermediate | I-5 | I-5 | I-5 | I-5 |
| Amount of Ag-containing | 174.9 | 174.5 | 174.8 | 174.2 |
| Intermediate [g] | | | | |
| Amount of Ag-complex | 77.829 | 77.607 | 77.758 | 77.500 |
| solution [g] | | | | |
| Amount of promoter | 3.280 | 3.271 | 3.277 | 3.266 |
| solution I [g] | | | | |
| Amount of promoter | 4.177 | 4.164 | 4.173 | 4.158 |
| solution II [g] | | | | |
| Cs-/W-/K-/Na-content in | 5.0/3.0/ | 4.05/3.0/ | 5.0/3.0/ | 4.05/3.0/ |
| promoter solution II [wt.-%] | 0.2/0.0 | 0.93/0.0 | 0.93/0.0 | 0.22/0.0 |
| Amount of promoter | 2.924 | 2.321 | 2.325 | 2.911 |
| solution III [g] | | | | |
| Amount of DI H₂O [g] | 0.0 | 0.632 | 0.617 | 0.0 |

The impregnated material was placed on a net forming 1 to 2 layers (about 100 to 250 g per calcination run). The net was subjected to 23 Nm³/h nitrogen flow (oxygen content: <20 ppm), wherein the gas flows were pre-heated to a temperature of 305 °C. The impregnated materials were heated up to a temperature of 290 °C at a heating rate of about 30 K/min and then maintained at 290 °C for 7 min to yield catalysts according to Table 6. The temperatures were measured by placing three thermocouples at 1 mm below the calcination net. Subsequently, the catalysts were cooled to ambient temperature by removing the catalyst bodies from the net using an industrial vacuum cleaner. All catalysts shown in Table 6 comprised an amount of lithium C_{Li} of 67.7 mmol/kg and an amount of cesium cs of 0.94 mmol/kg.

TABLE 6
(Ag-contents are reported in percent by weight of total catalyst, the amounts of
all further elements are reported in mmol/kg of total catalyst)

| Catalyst | Support | Ag * | c_{Re} | c_{W} | c_{Cs} | c_{Si} | IM_{K} ** | c_{K} *** | IM_{Na} ** | C_{Na} *** |
|---|---|---|---|---|---|---|---|---|---|---|
| 1-1 | A | 28.1 | 7.09 | 3.43 | 7.90 | 2.56 | 2.15 | 2.15-2.33 | 0.0 | 1.25 |
| 1-2 ^{#} | A | 28.9 | 6.28 | 3.02 | 9.03 | 2.53 | 5.19 | 5.19-5.37 | 1.77 | 3.00 |
| 1-3 ^{#} | A | 28.9 | 6.28 | 3.70 | 9.03 | 2.53 | 5.24 | 5.24-5.42 | 1.77 | 3.00 |
| 1-4 ^{#} | A | 28.9 | 7.89 | 3.70 | 8.65 | 2.53 | 5.24 | 5.24-5.42 | 1.77 | 3.00 |
| 1-5 ^{#} | A | 28.9 | 7.89 | 3.02 | 9.03 | 2.53 | 3.89 | 3.89-4.07 | 0.89 | 2.12 |
| 1-6 | B | 28.9 | 7.09 | 3.43 | 7.15 | <2.53 | 3.30 | 3.30-3.48 | 0.46 | 1.84 |
| 1-7 | B | 28.9 | 7.89 | 3.43 | 7.90 | <2.53 | 3.30 | 3.30-3.48 | 0.46 | 1.84 |
| 1-8 | B | 28.9 | 6.28 | 3.43 | 6.40 | <2.53 | 3.30 | 3.30-3.48 | 0.46 | 1.84 |
| 1-9 ^{#} | B | 28.9 | 6.28 | 3.43 | 7.90 | <2.53 | 3.30 | 3.30-3.48 | 0.46 | 1.84 |
| 1-10 ^{#} | B | 28.9 | 7.89 | 3.43 | 6.40 | <2.53 | 3.30 | 3.30-3.48 | 0.46 | 1.84 |
| 1-11 | B | 28.9 | 6.66 | 3.43 | 6.40 | <2.53 | 5.17 | 5.17-5.35 | 0.0 | 1.39 |
| 1-12 | B | 28.9 | 6.66 | 3.43 | 6.40 | <2.53 | 3.94 | 3.94-4.12 | 0.0 | 1.39 |
| 1-13 ^{#} | B | 28.9 | 7.89 | 3.43 | 7.90 | <2.53 | 7.06 | 7.06-7.24 | 0.0 | 1.39 |
| 1-14 | C | 28.9 | 7.09 | 3.43 | 7.15 | <2.53 | 3.30 | 3.30-3.48 | 0.0 | 1.39 |
| 1-15 | C | 28.9 | 7.09 | 3.43 | 7.15 | <2.53 | 3.30 | 3.30-3.48 | 0.46 | 1.84 |
| 1-16 | C | 28.9 | 7.89 | 3.43 | 7.90 | <2.53 | 3.30 | 3.30-3.48 | 0.0 | 1.39 |
| 1-17 | C | 28.9 | 6.28 | 3.43 | 6.40 | <2.53 | 3.30 | 3.30-3.48 | 0.0 | 1.39 |
| 1-18 | D | 27.3 | 7.09 | 3.43 | 7.15 | <2.59 | 3.17 | 3.17-3.35 | 0.0 | 1.42 |
| 1-19 | D | 27.3 | 7.89 | 3.43 | 7.90 | <2.59 | 3.17 | 3.17-3.35 | 0.0 | 1.42 |
| 1-20 ^{#} | D | 27.3 | 6.28 | 3.43 | 6.40 | <2.59 | 7.09 | 7.09-7.26 | 0.0 | 1.42 |
| 1-21 ^{#} | D | 27.3 | 6.28 | 3.43 | 7.90 | <2.59 | 7.09 | 7.09-7.26 | 0.0 | 1.42 |
| 1-22 ^{#} | D | 27.3 | 7.89 | 3.43 | 6.40 | <2.59 | 3.27 | 3.27-3.45 | 0.0 | 1.42 |

* Ag and all promoter values are calculated values
^{#} comparative example
** IMP_{K} and IMP_{Na} are understood to mean the amounts of potassium and sodium added during impregnation and do not include the amounts of potassium and sodium, respectively, comprised in the alumina support prior to impregnation
*** c_{K} and c_{Na} are understood to mean the total amount of potassium and sodium, respectively, in the catalyst. The amount of potassium derived from the supports was in the range of 0 to 0.183 mmol/kg, relative to the total weight of the catalyst.

### Example 3—Catalyst Testing

An epoxidation reaction was conducted in a vertically-placed test reactor constructed from stainless steel with an inner diameter of 6 mm and a length of 2.2 m. The reactor was heated using hot oil contained in a heating mantle at a specified temperature. All temperatures in Table 7 below refer to the temperature of the hot oil. The reactor was filled with 9 g of inert steatite balls (0.8 to 1.1 mm), onto which 27.0 g of crushed catalyst screened to a desired particle size of 1.12 to 1.4 mm were packed, and thereon an additional 29 g of inert steatite balls (0.8-1.1 mm) were packed. The inlet gas was introduced to the top of the reactor in a “once-through” operation mode.

The catalysts were charged into the reactor at a reactor temperature of 90 °C under nitrogen flow of 130 NL/h at a pressure of 1.5 bar absolute. Then, the reactor temperature was ramped up to 210 °C at a heating rate of 50 K/h and the catalysts were maintained under these conditions for 15 h. Subsequently, the nitrogen flow was substituted by a flow of 114 NL/h methane and 1.5 NL/h CO₂. The reactor was pressurized to 16 bar absolute. Subsequently, 30.4 NL/h ethylene and 0.8 NL/h of a mixture of 500 ppm ethylene chloride in methane were added. Then, oxygen was introduced stepwise to reach a final flow of 6.1 NL/h. At this point, the inlet composition consisted of 20 vol.-% ethylene, 4 vol.-% oxygen, 1 vol.-% carbon dioxide, and ethylene chloride (EC) moderation of 2.5 parts per million by volume (ppmv), with methane used as a balance at the total gas flow rate of 152.7 NL/h.

The reactor temperature was ramped up to 225 °C at a heating rate of 5 K/h, and afterwards to 240 °C at a heating rate of 2.5 K/h. The catalysts were maintained under these conditions for 135 hours. Afterwards, EC concentration was decreased to 2.2 ppmv, and the temperature was decreased to 225 °C. Subsequently, the inlet gas composition was gradually changed to 35 vol.-% ethylene, 7 vol.-% oxygen, 1 vol.-% carbon dioxide with methane used as a balance and a total gas flow rate of 147.9 NL/h. The temperature was adjusted to achieve an ethylene oxide (EO) concentration in the outlet gas of 3.05%. The EC concentration was adjusted to optimize the selectivity. Results of the catalyst tests are summarized in Table 7.

TABLE 7
Results of catalyst tests.

| | | | | time on stream | | time on stream | | time on stream | |
|---|---|---|---|---|---|---|---|---|---|
| | | | | ^{§} = 11 days | | ^{§} = 25 days | | ^{§} = 28 days | |
| | | | | S | T | S | T | S | |
| Test | Catalyst | P_{A} | P_{B} | [%] | [°C] | [%] | [°C] | [%] | ΔS₂₈₋₂₅ |
| 2-1 | 1-1 | 0.647 ± 0.002 | 0.443 ± 0.012 | 88.5 | 233 | 88.8 | 234 | 88.7 | −0.1 |
| 2-2 | 1-2 ^{#} | 0.441 ± 0.002 | 0.918 ± 0.010 | 81 * | 250 * | — ** | — | — | — |
| 2-3 | 1-3 ^{#} | 0.459 ± 0.001 | 0.923 ± 0.010 | 82.2 * | 245 | — | — | — | — |
| 2-4 | 1-4 ^{#} | 0.568 ± 0.002 | 0.963 ± 0.011 | 87.4 | 239 | 86.7 | 246 | 86.6 | −0.1 |
| 2-5 | 1-5 ^{#} | 0.567 ± 0.002 | 0.675 = 0.010 | 86.1 | 232 | 88.1 | 239 | — | — |
| 2-6 | 1-6 | 0.660 ± 0.002 | 0.732 ± 0.013 | 88.4 | 233 | 89.2 | 232 | 89.2 | 0.0 |
| 2-7 | 1-7 | 0.664 ± 0.002 | 0.662 ± 0.012 | 88.2 | 234 | 88.9 | 234 | 88.9 | 0.0 |
| 2-8 | 1-8 | 0.655 ± 0.002 | 0.818 ± 0.014 | 88.2 | 233 | 88.8 | 234 | — | — |
| 2-9 | 1-9 ^{#} | 0.553 ± 0.002 | 0.662 ± 0.012 | 85.6 | 229 | — | — | — | — |
| 2-10 | 1-10 ^{#} | 0.787 ± 0.003 | 0.818 ± 0.014 | 87 | 232 | — | — | — | — |
| 2-11 | 1-11 | 0.649 ± 0.003 | 1.039 ± 0.014 | 88.5 | 235 | 89.1 | 235 | 89.0 | −0.1 |
| 2-12 | 1-12 | 0.681 ± 0.003 | 0.847 ± 0.014 | 88.1 | 234 | 89.1 | 235 | 89.1 | 0.0 |
| 2-13 | 1-13 ^{#} | 0.596 ± 0.002 | 1.080 ± 0.012 | 86.0 | 233 | — | — | — | — |
| 2-14 | 1-14 | 0.672 ± 0.003 | 0.668 ± 0.013 | 88.8 | 235 | — | — | — | — |
| 2-15 | 1-15 | 0.660 ± 0.003 | 0.732 ± 0.013 | 88.7 | 236 | — | — | — | — |
| 2-16 | 1-16 | 0.675 ± 0.002 | 0.604 ± 0.012 | 89.1 | 235 | 89.7 | 238 | 89.5 | −0.2 |
| 2-17 | 1-17 | 0.668 ± 0.003 | 0.747 ± 0.014 | 89.1 | 236 | 89.7 | 239 | 89.8 | 0.1 |
| 2-18 | 1-18 | 0.674 ± 0.002 | 0.655 ± 0.013 | 89.2 | 239 | 89.9 | 241 | — | — |
| 2-19 | 1-19 | 0.677 ± 0.002 | 0.592 ± 0.012 | 88.7 | 242 | 89.5 | 245 | — | — |
| 2-20 | 1-20 ^{#} | 0.576 ± 0.002 | 1.344 ± 0.014 | 82.4 * | 250 * | — | — | — | — |
| 2-21 | 1-21 ^{#} | 0.496 ± 0.002 | 1.088 ± 0.012 | 77.3 * | 250 * | — | — | — | — |
| 2-22 | 1-22 ^{#} | 0.802 ± 0.003 | 0.748 ± 0.014 | 87.5 | 237 | 88.1 | 240 | — | — |

^{#} comparative example
^{§} time on stream: determined from the point of time of oxygen introduction
* target EO-outlet of 3.05% could not be achieved
** “—” = not determined

It is evident that catalysts having a value of P_{A} within a specific range exhibit a higher selectivity after 11 days and after 25 days than comparative catalysts. These findings are depicted in FIGS. 2 and 3.

It is moreover evident that catalysts having a value of P_{B} within a specific range exhibit a higher stability of ethylene oxide selectivity than catalysts outside this range, as is evident from the value of ΔS₂₈₋₂₅, i.e., the difference in selectivity after 28 days and after 25 days on stream. These findings are depicted in FIGS. 4 and 5.

1.-15. (canceled)

16. An epoxidation catalyst comprising silver, cesium, rhenium and tungsten deposited on a low silicon alpha-alumina support which comprises at least 50 wt.-% of alpha-alumina, and an amount of silicon c_{S-Si} of at most 8.9 mmol per kg of support,
the catalyst further comprising potassium and optionally sodium, wherein the catalyst comprises
25 to 50 wt.-% of silver, relative to the weight of the catalyst,
an amount of cesium c_{Cs} of at least 6.0 mmol per kg of catalyst,
an amount of rhenium c_{Re} of at least 6.0 mmol per kg of catalyst,
an amount of tungsten c_{W} of at least 3.0 mmol per kg of catalyst,
an amount of potassium c_{K} of at least 3.0 mmol per kg of catalyst, and
an amount of sodium c_{Na} of at most 4.0 mmol per kg of catalyst; and
wherein the value of P_{A}
$${P}_{A} = \frac{{c}_{Re} + ( 0.5 \times {c}_{W} )}{( 1.5 \times {c}_{C s} ) + ( 0.5 \times ( {c}_{K} + {c}_{N a} ) )}$$
is in the range of 0.62 to 0.76, the elemental composition of the catalyst support and the catalyst being determined by elemental analysis via inductively coupled plasma optical emission spectroscopy.
The catalyst according to claim 16, wherein the value of P_{B}
$${P}_{B} = \frac{{c}_{K} + {c}_{Na}}{{c}_{C s}}$$
is in the range of 0.62 to 0.94.

17. The catalyst according to claim 16, comprising an amount of cesium c_{Cs} of 6.0 to 9.0 mmol per kg of catalyst.

18. The catalyst according to claim 16, comprising an amount of rhenium c_{Re} of 6.0 to 9.0 mmol per kg of catalyst.

19. The catalyst according to claim 16, comprising an amount of tungsten c_{W} of 3.0 to 5.0 mmol per kg of catalyst.

20. The catalyst according to claim 16, comprising an amount of potassium cx of 3.0 to 6.0 mmol per kg of catalyst.

21. The catalyst according to claim 16, comprising an amount of sodium c_{Na} at most 3.0 mmol per kg of catalyst.

22. The catalyst according to claim 16, wherein the alumina support comprises at least 80 wt.-% alpha-alumina.

23. The catalyst according to claim 16, wherein the alpha-alumina support is a tableted alpha-alumina support.

24. The catalyst according to claim 16, wherein the alpha-alumina support has a BET surface area of 0.5 to 3.0 m²/g.

25. The catalyst according to claim 16, wherein the catalyst has a total Hg pore volume of 0.2 to 1.0 mL/g as determined by mercury porosimetry.

26. A process for preparing an epoxidation catalyst as defined in claim 16, comprising
i) impregnating a low silicon alpha-alumina support with a silver impregnation solution; and
ii) subjecting the impregnated refractory support to a calcination process;
wherein steps i) and ii) are optionally repeated, and at least one silver impregnation solution comprises rhenium, tungsten, cesium, potassium, and optionally sodium.

27. The process according to claim 27, wherein the support comprises an amount of silicon c_{S-Si} of at most 5.4 mmol per kg of support.

28. The process according to claim 27, wherein the support comprises
an amount of potassium c_{S-K} of at most 3.0 mmol per kg of support, and
an amount of sodium c_{S-Na} of at most 4.0 mmol per kg of support.

29. A process for producing ethylene oxide by gas-phase oxidation of ethylene, comprising reacting ethylene and oxygen in the presence of an epoxidation catalyst according to claim 16.
